(19) United States
(12) Patent Application Publication
(10) Pub. No.: US 2010/0029004 A1
(43) Pub. Date: Feb. 4, 2010

(54) DURATION AND ENVIRONMENTAL MONITORING COMPOSITIONS, DEVICES METHODS FOR PREPARATION AND SYSTEMS

(76) Inventor: Hans O. Ribi, Hillsborough, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/510,814

(22) Filed: Jul. 28, 2009

Related U.S. Application Data
(60) Provisional application No. 61/085,750, filed on Aug. 1, 2008.

(51) Int. Cl. G01N 21/75; C08L 49/00; G01N 33/02

(52) U.S. Cl. 436/20; 524/550; 436/164

(57) ABSTRACT

Duration monitoring co-topo-polymeric compositions and methods for making and using the same are provided. Indicator compositions of the invention include an organic polymer and undergo a color change, which may be reversible or irreversible, in a time dependent manner following an initiation event. Also provided are indicator devices that include the indicator compositions of the invention. The compositions of the invention find use in a variety of different applications.

### CROSS-REFERENCE TO RELATED APPLICATIONS

Pursuant to 35 U.S.C. §119 (e), this application claims priority to the filing dates of U.S. Provisional Patent Application Ser. No. 61/085,750 filed Aug. 1, 2008; the disclosure of which application is herein incorporated by reference.

### INTRODUCTION

There are many instances in which knowledge of a time parameter of a product is desirable. For example, with perishable products, it is desirable to know one or more time parameters, such as time since fabrication of the product, time since exposure of the product to a given condition of interest, e.g., conditions that might compromise the quality of the product, etc. While a variety of different approaches have been developed to provide such time parameters, there is continued interest in the development of yet new approaches.

### SUMMARY

Duration monitoring compositions and methods for making and using the same are provided. Indicator compositions of the invention include an organic polymerizable components, where the compositions undergo a color change, which may be reversible or irreversible, in a time dependent manner, such as in response to applied conditions. The duration monitoring compositions of the invention may be associated with a product of interest and provide to a user an indication of a temporal parameter of the associated product, such as a time since an event in the product's lifecycle, e.g., fabrication or some later event, such as exposure to an adverse condition (e.g., temperature), product life expectancy, etc. In some instances, the duration monitoring compositions are configured to provide to a user a temporal parameter that changes depending on ambient conditions experienced by a product during the product's lifetime, such as whether the associated product was exposed to an adverse condition, including how long it was exposed to the adverse condition. Also provided are indicator devices that include the indicator compositions of the invention. The compositions of the invention find use in a variety of different applications.

### DETAILED DESCRIPTION

Aspects of the invention include duration monitoring compositions. Duration monitoring compositions of the invention include at least a first polymerizable monomer, wherein the duration monitoring composition is configured to change color at a predetermined time after polymerization initiation of the composition. Polymerization initiation may occur as a result of the composition being exposed to ambient conditions, being exposed to a polymerizing agent, e.g., UV light, etc. In some instances, the duration monitoring compositions include, in addition to the first monomer, at least one of if not both of a second monomer that is distinct from the first monomer; and an effector compound. In some embodiments, the first and second monomers are diacetylenic monomers. Where desired, the first and second diacetylenic monomers may differ from each other in one or more of: monomer chain length, head-group structure, bond positioning, appendages, chirality, related features, and/or combinations thereof. In some instances, each of the first and second diacetylenic monomers is independently used to develop a color.

Duration monitoring compositions of the invention may be configured to initiate polymerization upon exposure to ambient conditions or upon application of a stimulus to the composition, e.g., by removing a barrier and/or exposing the composition to UV radiation.

The duration monitoring composition may be applied directly to an object to be monitored or may be present on a substrate, such as a label, e.g., where the label is applied to an object to be monitored.

In some embodiments, the substrate may itself be a sensor.

Duration monitoring compositions of the invention may include a number of additional components, such as but not limited to: thermochromic dyes, cholesteric liquid crystal, oils, luminescent or fluorescent pigments, etc. In some instances, one or more components of the composition are encapsulated. In some instances, the duration monitoring compositions may further include a nucleator.

Aspects of the invention further include duration indicator devices, where the indicator devices include a duration monitoring composition, e.g., as summarized above. In some embodiments of indicator devices, the duration monitoring composition is present on a surface of a solid support. Where desired, the indicator devices may include two or more distinct duration monitoring compositions on the surface of the solid support. In certain of these embodiments, the two or more distinct duration monitoring compositions may change color at a different time following initiation of polymerization. Where desired, the devices may further include a sensor that is not a duration monitoring composition. Devices of interest may include the duration monitoring composition in a machine readable format, e.g., a barcode.

Also provided are methods of producing duration monitoring compositions. Such methods may include providing a duration monitoring precursor composition that includes a first monomer and at least one of, if not both of, a second monomer that is distinct from said first monomer; and an effector compound. Following provision of the precursor composition, the methods may include setting the duration monitoring precursor composition into a solid product to produce the duration monitoring composition. In some instances, the methods may include placing the duration monitoring precursor composition onto a surface of a solid support prior to setting, where the placing may include printing.

Aspects of the invention further include methods of determining a time parameter of a product, where time parameters of interest include age of product, time since a product was exposed to a condition of interest, such as elevated temperatures, etc. Such methods include identifying a color change in a duration monitoring composition (e.g., as described above) associated with the product, and determining the time parameter of interest from the occurrence of the identified color change. The time parameter may be the duration of time since polymerization of the composition was initiated. In some instances, the method is a method of determining whether the product has been exposed to an ambient condition of interest, e.g., an elevated temperature, light, etc. In certain embodiments, the ambient condition initiates polymerization of the composition.

Such methods may be employed with a variety of different products. Products of interest include perishable products, such as but not limited to: consumer product, e.g., foods, such as meats (e.g., fish, shellfish, beef, pork, poultry, game, etc.), eggs, dairy products, produce (including vegetables and fruits), packaged prepared foods (such as sandwiches), etc. In some embodiments, the product is a beverage. Beverages of interest include, but are not limited to: alcoholic beverages (such as wine and beer), non-alcoholic beverages, such as those that include a dairy component and a fruit juice, etc.

Additional products of interest include, but are not limited to: medicines, chemicals, biologics (e.g., blood), fragrances, inhalables, inventoried products, etc.

The above aspects are described in greater detail below.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Duration Monitoring Compositions, Devices Including the Same and Methods of Fabrication

Compositions that change color in a time dependent manner as measured from an initiating or actuating starting point, where the time to color change may be a short, medium or long duration, are provided. The compositions are co-topo-polymeric compositions that include at least 1 monomer which undergoes a polymerization event to produce a color change in a time dependent manner. Compositions of interest may include two or more monomer analogs, e.g., that differ structurally in one or more key elements, combinations of elements, etc., where compositions may also include selected ratios between analogs, and may include an effector additives used during co-crystallization and latency, to provide for the desired duration monitoring composition. In some instances, a desired short, medium, or long duration color change composition is provided by using co-topo-polymeric compositions that includes at least 1 monomer and a co-mixing analog that is different structurally in one or more key elements or combinations of elements, as well as by selecting proper ratios between analogs, and in some instances including an effector additive used during co-crystallization of the composition components and/or a barrier for controlling the time dependent color change and therefore the duration monitoring ability of the composition. Color development duration transitions can be irreversible or reversible.

As reviewed in greater detail below, based on their formulations, the compositions have a known or predetermined color change characteristic which occurs at a known or predetermined time following an actuation or initiation of polymerization of one or more components of the composition. The compositions may be formulated such that initiation occurs in response to an applied stimulus (e.g., removing a barrier, exposing to polymerization conditions for a predetermined period of time) or may occur automatically, e.g., where the compositions are configured for autopolymerization, as desired. Examples of different formats for initiation are described in greater detail below.

### Diacetylenic Monomers

In certain embodiments, duration monitoring compositions of the invention include two or more diacetylene monomer analogs that differ from each other in one or more ways, including but not limited to monomer chain length, head-group structure, bond positioning, appendages, chirality, related features, and/or combinations thereof. In preparing such compositions, the two or more diacetylenic monomers can be admixed in a dissolved state and co-crystallized to produce the duration monitoring composition, where the duration monitoring composition undergoes co-topo-polymerization in the solid state in a time-dependent manner. With mixed systems, the monomers therein may or may not be independently polymerizable. However, the resulting combination is polymerizable resulting in color development during duration monitoring, i.e., following an initial event, such as fabrication of the composition or a time subsequent thereto, e.g., removing a barrier, exposing to a certain temperature for a sufficient period of time, etc.

Mixed compositions can be formulated to significantly alter the properties of the final system compared with the properties of a pure analog utilized to formulate the final system. By way of example but not limitation, two low duration monomers such as the carboxylic acid of 10,12-heneicosadiynoic acid and amide of 10,12-heneicosadiynoic amide; carboxylic acid of 10,12-tricosadiynoic acid and amide of 10,12-tricosadiynoic amide; carboxylic acid of 10,12-pentacosadiynoic acid and amide of 10,12-pentacosadiynoic amide; or bis diamide forms of 10,12-heneicosadiynoic acid and bis diamide forms of 10,12-tricosadiynoic amide can be mixed in varying ratios to achieve discrete threshold transition in the monomeric state, discrete polymerization timing, discrete color change duration transitions, and selective sensitivity ranges to other color change stimuli including, but not limited to temperature, friction, chemical triggering, solvent triggering, and/or other environmental stimuli. Ratios can range from a fraction of a mole percent of one component to a fraction of a mole percent of the other component. Both eutectic and non-eutectic mixtures can be achieved with accurate measurement. The color change triggering duration can be lowered less than 1 minute to greater than one year compared with duration transitions of a single component (e.g., only the acid form of 10,12-pentacosadiynoic acid).

Asymmetric acyl:non-acyl groups appended to each end of the diactylenic group are of particular interest due to the versatile nature and flexibility of corresponding analogs that can be generated by chemical modification and importantly due to the ability to modify the color development duration transition of a particular analog set.

Of interest are dual monomer precursor compositions that are made up of two different carboxylic acid diacetylenic monomers, where specific examples of such precursor compositions are shown in Table 1, below.

TABLE 1
Dual Monomer Precursor Carboxylic Acid Diacetylene
Monomer Compositions

| | | |
|---|---|---|
| 10,12-C18:10,12-C19 | 10,12-C19:10,12-C20 | 10,12-C20:10,12-C21 |
| 10,12-C18:10,12-C20 | 10,12-C19:10,12-C21 | 10,12-C20:10,12-C22 |
| 10,12-C18:10,12-C21 | 10,12-C19:10,12-C22 | 10,12-C20:10,12-C23 |
| 10,12-C18:10,12-C22 | 10,12-C19:10,12-C23 | 10,12-C20:10,12-C24 |
| 10,12-C18:10,12-C23 | 10,12-C19:10,12-C24 | 10,12-C20:10,12-C25 |
| 10,12-C18:10,12-C24 | 10,12-C19:10,12-C25 | 10,12-C20:10,12-C26 |
| 10,12-C18:10,12-C25 | 10,12-C19:10,12-C26 | 10,12-C20:10,12-C27 |
| 10,12-C18:10,12-C26 | 10,12-C19:10,12-C27 | 10,12-C20:10,12-C28 |
| 10,12-C18:10,12-C27 | 10,12-C19:10,12-C28 | 10,12-C20:10,12-C29 |
| 10,12-C18:10,12-C28 | 10,12-C19:10,12-C29 | 10,12-C20:10,12-C30 |
| 10,12-C18:10,12-C29 | 10,12-C19:10,12-C30 | |
| 10,12-C18:10,12-C30 | | |
| 10,12-C21:10,12-C22 | 10,12-C22:10,12-C23 | 10,12-C23:10,12-C24 |
| 10,12-C21:10,12-C23 | 10,12-C22:10,12-C24 | 10,12-C23:10,12-C25 |
| 10,12-C21:10,12-C24 | 10,12-C22:10,12-C25 | 10,12-C23:10,12-C26 |
| 10,12-C21:10,12-C25 | 10,12-C22:10,12-C26 | 10,12-C23:10,12-C27 |
| 10,12-C21:10,12-C26 | 10,12-C22:10,12-C27 | 10,12-C23:10,12-C28 |
| 10,12-C21:10,12-C27 | 10,12-C22:10,12-C28 | 10,12-C23:10,12-C29 |
| 10,12-C21:10,12-C28 | 10,12-C22:10,12-C29 | 10,12-C23:10,12-C30 |
| 10,12-C21:10,12-C29 | 10,12-C22:10,12-C30 | |
| 10,12-C21:10,12-C30 | | |
| 10,12-C24:10,12-C25 | 10,12-C25:10,12-C26 | 10,12-C26:10,12-C27 |
| 10,12-C24:10,12-C26 | 10,12-C25:10,12-C27 | 10,12-C26:10,12-C28 |
| 10,12-C24:10,12-C27 | 10,12-C25:10,12-C28 | 10,12-C26:10,12-C29 |
| 10,12-C24:10,12-C28 | 10,12-C25:10,12-C29 | 10,12-C26:10,12-C30 |
| 10,12-C24:10,12-C29 | 10,12-C25:10,12-C30 | |
| 10,12-C24:10,12-C30 | | |
| 10,12-C27:10,12-C28 | 10,12-C28:10,12-C29 | |
| 10,12-C27:10,12-C29 | 10,12-C28:10,12-C30 | |
| 10,12-C27:10,12-C30 | | |

Also of interest are triple monomer precursor compositions that are made up of three different carboxylic acid diacetylenic monomers, where specific examples of such precursor compositions are shown in Table 2, below.

TABLE 2
Triple Monomer Precursor Composition of Carboxylic Acid Diacetylenic
Monomers

| | | |
|---|---|---|
| 10,12-C18 | 10,12-C19 | 10,12-C20 |
| 10,12-C19 | 10,12-C20 | 10,12-C21 |
| 10,12-C20 | 10,12-C21 | 10,12-C22 |
| 10,12-C21 | 10,12-C22 | 10,12-C23 |
| 10,12-C22 | 10,12-C23 | 10,12-C24 |
| 10,12-C23 | 10,12-C24 | 10,12-C25 |
| 10,12-C24 | 10,12-C25 | 10,12-C26 |
| 10,12-C25 | 10,12-C26 | 10,12-C27 |
| 10,12-C26 | 10,12-C27 | 10,12-C28 |
| 10,12-C27 | 10,12-C28 | |
| 10,12-C28 | 10,12-C29 | |
| 10,12-C29 | 10,12-C30 | |

Also of interest are dual monomer precursor compositions that include an amide diacetylenic monomer and a carboxylic acid diacetylenic monomer, e.g., as exemplified in Table 3, below.

TABLE 3
Dual Monomer Precursor Composition
ET = Ethyl

| | | |
|---|---|---|
| EtNH-10,12-C18:10,12-C19 | EtNH-10,12-C19:10,12-C20 | EtNH-10,12-C20:10,12-C21 |
| EtNH-10,12-C18:10,12-C20 | EtNH-10,12-C19:10,12-C21 | EtNH-10,12-C20:10,12-C22 |
| EtNH-10,12-C18:10,12-C21 | EtNH-10,12-C19:10,12-C22 | EtNH-10,12-C20:10,12-C23 |
| EtNH-10,12-C18:10,12-C22 | EtNH-10,12-C19:10,12-C23 | EtNH-10,12-C20:10,12-C24 |
| EtNH-10,12-C18:10,12-C23 | EtNH-10,12-C19:10,12-C24 | EtNH-10,12-C20:10,12-C25 |
| EtNH-10,12-C18:10,12-C24 | EtNH-10,12-C19:10,12-C25 | EtNH-10,12-C20:10,12-C26 |
| EtNH-10,12-C18:10,12-C25 | EtNH-10,12-C19:10,12-C26 | EtNH-10,12-C20:10,12-C27 |
| EtNH-10,12-C18:10,12-C26 | EtNH-10,12-C19:10,12-C27 | EtNH-10,12-C20:10,12-C28 |
| EtNH-10,12-C18:10,12-C27 | EtNH-10,12-C19:10,12-C28 | EtNH-10,12-C20:10,12-C29 |
| EtNH-10,12-C18:10,12-C28 | EtNH-10,12-C19:10,12-C29 | EtNH-10,12-C20:10,12-C30 |
| EtNH-10,12-C18:10,12-C29 | EtNH-10,12-C19:10,12-C30 | |
| EtNH-10,12-C18:10,12-C30 | | |
| EtNH-10,12-C21:10,12-C22 | EtNH-10,12-C22:10,12-C23 | EtNH-10,12-C23:10,12-C24 |
| EtNH-10,12-C21:10,12-C23 | EtNH-10,12-C22:10,12-C24 | EtNH-10,12-C23:10,12-C25 |
| EtNH-10,12-C21:10,12-C24 | EtNH-10,12-C22:10,12-C25 | EtNH-10,12-C23:10,12-C26 |
| EtNH-10,12-C21:10,12-C25 | EtNH-10,12-C22:10,12-C26 | EtNH-10,12-C23:10,12-C27 |
| EtNH-10,12-C21:10,12-C26 | EtNH-10,12-C22:10,12-C27 | EtNH-10,12-C23:10,12-C28 |
| EtNH-10,12-C21:10,12-C27 | EtNH-10,12-C22:10,12-C28 | EtNH-10,12-C23:10,12-C29 |
| EtNH-10,12-C21:10,12-C28 | EtNH-10,12-C22:10,12-C29 | EtNH-10,12-C23:10,12-C30 |
| EtNH-10,12-C21:10,12-C29 | EtNH-10,12-C22:10,12-C30 | |
| EtNH-10,12-C21:10,12-C30 | | |
| EtNH-10,12-C24:10,12-C25 | EtNH-10,12-C25:10,12-C26 | EtNH-10,12-C26:10,12-C27 |
| EtNH-10,12-C24:10,12-C26 | EtNH-10,12-C25:10,12-C27 | EtNH-10,12-C26:10,12-C28 |
| EtNH-10,12-C24:10,12-C27 | EtNH-10,12-C25:10,12-C28 | EtNH-10,12-C26:10,12-C29 |
| EtNH-10,12-C24:10,12-C28 | EtNH-10,12-C25:10,12-C29 | EtNH-10,12-C26:10,12-C30 |
| EtNH-10,12-C24:10,12-C29 | EtNH-10,12-C25:10,12-C30 | |
| EtNH-10,12-C24:10,12-C30 | | |
| EtNH-10,12-C27:10,12-C28 | EtNH-10,12-C28:10,12-C29 | |
| EtNH-10,12-C27:10,12-C29 | EtNH-10,12-C28:10,12-C30 | |
| EtNH-10,12-C27:10,12-C30 | | |

Also of interest are triple monomer precursor compositions that include an amide diacetylenic monomer and two different carboxylic acid diacetylenic monomers, e.g., as exemplified in Table 4, below.

TABLE 4
Triple Monomer Precursor Composition of Three Different Amide
and Carboxylic Acid Diacetylenic Monomers

| | | |
|---|---|---|
| EtNH-10,12-C18 | EtNH-10,12-C19 | EtNH-10,12-C20 |
| 10,12-C19 | 10,12-C20 | 10,12-C21 |
| 10,12-C20 | 10,12-C21 | 10,12-C22 |
| EtNH-10,12-C21 | EtNH-10,12-C22 | EtNH-10,12-C23 |
| 10,12-C22 | 10,12-C23 | 10,12-C24 |
| 10,12-C23 | 10,12-C24 | 10,12-C25 |
| EtNH-10,12-C24 | EtNH-10,12-C25 | EtNH-10,12-C26 |
| 10,12-C25 | 10,12-C26 | 10,12-C27 |
| 10,12-C26 | 10,12-C27 | 10,12-C28 |
| EtNH-10,12-C27 | EtNH-10,12-C28 | |
| 10,12-C28 | 10,12-C29 | |
| 10,12-C29 | 10,12-C30 | |

The above tables merely provide examples of the different combinations of diacetylenic monomers that may be present in a given indicator precursor composition.

Of particular interest are end-of-use indicators (such as indicator compositions that impart to a user information with respect to when a particular product has reached its useful lifetime) that can be manipulated in durations up and down in time by shifting mixed analog systems of corresponding intermolecular interactions whereby the duration dependent color development transition of a composition can be shifted to between 10 seconds to over 100 days depending on the structural ratios and the modification employed.

Further duration shifting can be accomplished by admixing selected ratios of different mixed chain compositions correspondingly to change the initially duration of pure forms compared to unmixed compositions. The effective combination of chemical structure and mixed compositions can bring an initial composition structure from a duration color transition of greater than only minutes to over months for the color generation process.

### Duration Monitoring and End-of-Use Time Frame Manipulation:

Duration dependent/end-of-use co-topo-polymeric compositions disclosed herein can be adjusted to exhibit an altered and accelerated or decelerated degree of time dependent color change, as compared to a corresponding pure composition. Doping one component of a duration monitoring co-topo-polymeric component with another like-kind component promotes the degree of polymerization color change development compared with pure individual compositions.

Admixed duration or end-of-use monitoring chemistry components can be in pairs or more complex mixtures of 3 or more components. Mixed compositions of interest are blends of from 2 to more than 50 individual monomer types, such as from 2 to 10 types, including from 2 to 4 different analog types.

Ratios of two components can range from 0.001% of a first component to 99.999% of a second component, such as from 0.01 of a first component to 99.99% of a second component, including from 0.1% first component to 99.9% of a second component, e.g., from 1% of a first component to 99% of a first component. The exact ratio of each component will be dictated by the desired duration setting and characteristics of the intended product application for the composition.

For hydrocarbon based structures, single side-chain compounds, dual chain compounds or higher order chain-containing analogs can be used alone or in combinations here within in the prescribed ratios. The mixed duration monitoring co-topo-polymeric composition containing only single chain monomers or mixtures of multiple chain monomers with single chain monomers will be dictated by the product application of interest.

Of particular interest for increasing color development duration time-frames is the use of diacetylenic monomeric analogs terminated with carboxylic acid moieties. Of particular interest for decreasing the color development duration time-frame is the use of dicacetylenic monomeric analogs terminated with amide moieties. Mixed co-topo-polymeric compositions for duration indication can be formulated using mixed compositions of a particular moiety or of differentiated moieties.

In some instances, duration monitoring compositions are configured for detection of triggering elements alternative to temperature. Lipids, oils fats, greases, selected reactive chemistries, ionic strength changes, solvents, gases, pH changes and the like can be used alone or in combination with a duration time monitoring event as triggering compositions to induce a color change event in a duration monitoring co-topo-polymeric composition. Triggering agents can initiate a color change event in the co-topo-polymeric composition in from a 1:1000,000 molar ratio to a 1:100,000 molar ratio between the triggering composition and the duration monitoring composition, such as between 1:10,000 and 10,000:1, and including between 1:1,000 and 1,000:1, e.g., between 1:100 and 100:1 will find effect.

Multi-parameter indicators can be formulated and based on duration monitoring compositions that incorporate a co-topo-polymeric composition for temperature monitoring and at least one or more of a second indicating means for determining the quality or state of a product being monitored. Secondary indicator means in a multi-parameter indicator can include but are not limited to sensors that measure: temperature, food breakdown bi-products such as those from meat spoilage, pressure utilized in ultra-intense sterilization processing equipment, product replacement cycles where either manufactures or consumers can tell when a product or product element should be replaced, high temperature indicators for measuring food cooking temperatures, freshness of consumable products whether oral or inhalable, pH for monitoring food processing conditions, gases emitted from a product for monitoring food or other item processing or storage states, vacuum sealed thermally insulated sensors, and the like.

During fabrication, components and precursors of duration monitoring and suggested end-of-use indication compositions can be applied neat, in oil forms, in microencapsulated forms, in aqueous coating matrix compositions, in solvent-based coating compositions, in emulsions, with protective over-coatings, in soluble plastic matrix resins, in wax matrix compositions, a range of different organic or polymeric matrix compositions, rubbers, thermal set resins, epoxies, varnishes, printing inks and binders and the like.

### Machine-Readable Chemistries and Device Configurations:

Machine-readable chemistry and device configurations can include, but are not limited to various printed barcodes, Interactive barcodes, abuse security barcodes; 1D, 2D, and 3D; barcodes holographic barcodes, vision imaging systems, transient barcodes, time-only barcodes, freshness indicating barcodes, shape memory bar codes, and a variety of other applications and formats.

Compositions herein can be formulated and utilized in a variety of visual, scanning, imaging, and machine readable processes as they relate to time monitoring algorithms. Messages or codes can be made to appear or disappear; parts or elements of graphics, symbols or codes can be utilized to make the element, graphic, or code un-discernable or unrecognizable until that portion of the medium has changed with temperature or the like.

Duration monitoring/end-of-use monitoring and indicating compositions can be utilized in both visual and machine aided formats. Visual readings are made with distinct visual determination of a threshold color change that occurs. Machine aided formats are made using an optical or electrical interpreted change in a color hue or conductive characteristic in a composition that undergoes a state threshold change. By way of example, but not limitation, a composition can be printed or formulated in a machine viewable format. A measurable reading may be taken of an initial calorimetric state. A second or sequential reading can be measured as threshold state occurs. During the transition from one state to another state, an instrumented reading can be registered. The threshold transition can be measured against a calibrated reading such that the degree or magnitude of the composition state change can be recorded and monitored. Recorded and monitored machine measurements can be displayed by instrumentation utilized in the machine aided format.

Machine readable/responsive barcodes can be utilized for determining the presence of or responding to a duration fluctuation, visible light, ultra-violet light, irradiation for applications such as food sterilization including gamma and cobalt 60 irradiation levels, hydration, pressure changes, high pressure events including high pressure sterilization, contaminations such a heavy metal contamination, alcohol levels, poisons, chemical sensing, biological compositions, chemical reagents, non-specific analyte binding, specific analyte binding, gases, physical and mechanical responses, UV intensity, light intensity, sanitization conditions, mechanical stress conditions, pressurization formats, oxidation state, optical bleaching, end-of-use indication, time, time and temperature, free radical content, hydration state, skin care health, medical sterilization, clinical health status, indicating sensors on food storage containers medical status, security applications, anti-tampering applications, and any of a number of other measurable indicia.

Machine readable codes for indicating time duration for product shelf-life and use indication can be accomplished using sensing compositions that shift spectrally in response to ambient conditions and product storage. Of interest are time only indicating barcodes that show machine readable expiration of product.

Of interest in certain instances are barcodes embedded or obscured in conjunction with duration monitoring layer/material that is selectively revealed upon triggering at set points of co-topo-chemical agent.

A range of barcode languages can be utilized that can be partially of fully associated with a determined composition and therefore act as a machine readable indication means to measure and report the selective functionality intended to comprise the composition used for indication. Barcode types include, but are not limited to any language, a wide range in size and numbers of character, as well as the barcode language of interest: 39, 93, 128A, 128B, 128C.

A standard barcode or UPC code can be obscured, coated, embedded in or over-laid by a mixed or single component duration monitoring composition of the invention. Part of the standard bar code can be clearly visible at the beginning of reading so as to generate an initial starting parameter set. Selective portions of the barcode can be covered by discrete duration monitoring compositions that can be set to change color at pre-determined timing exposures and/or development of intensified color over the duration where by a given code becomes readable prior to it not being readable. As the barcode is placed on a product type at a beginning time frame the duration monitoring chromic change composition can be activated. On activation, pre-determined elements of the code will be obscured by the optical density of the duration monitoring composition. The optical density of the barcode will be set such that a barcode reader will not be able register the obscured portion/bars that represent a specific code sequence. As the barcode/product is held in time and as pre-selected timing compositions are achieved and exposed, a pre-determined section of bar code will be revealed (reversibly or irreversibly depending on the nature of the chromic change agent selected). As each timing threshold is achieve during the timing exposure process, each pre-determined/coated barcode region will be come machine readable.

Non-readable or partially readable barcodes utilizing single or mixed composition as the obscuring agent are readily scanned for activity or inactivity in part or in whole.

Polydiacetylenes and other blue/black bar codes provide a unique optical masking characteristic that makes partially readable of fully non-readable part or all of the modified bar code. In addition, the transition of a blue/black polydiacetylenic compound to a red or orange hue including but not limited to light pink to dark red hues, provides for high optical readability by most commercial barcode readers since the red, orange, pink or related hues are optically transparent to the red light sources utilized in standard barcode readers.

Readable barcode languages include but are not limited to: Morovia Code 25, 11, 12B. 139. UPC-A, UPC-E, EAN-8, EAN-13, code 128b, USS 39, USD 3, 3 of 9 code, code 39. hibcc. Java applet, logmars, full, symbology, industry 2 of 5, discrete, self checking codes, one-dimensional barcodes, two-dimensional barcodes, three-dimensional barcodes, halo-graphic barcodes, luminescent barcodes, and the like.

Configurations of interest include, but are not limited to: Off to On switching barcodes: On to off switching barcodes: Codes 39 and 93 for embedded thermal messaging, etc. A variety of barcode geometries may be employed, including but not limited to: planar, curved, round etc. Of interest are barcodes for thermal delay for time coding; freshness indicating barcodes; time-only indicating barcodes, etc.

Additional details regarding machine readable formats of interest are disclosed in U.S. application Ser. No. 12/505,405 filed on Jul. 17, 2009, the disclosure of which is herein incorporated by reference.

### Duration Delay and Related Product Mechanisms:

Often it will be important to utilize product devices and application where there will be either no time delay history mechanism in the color change process embedded in a device, a minimal delay mechanism or history in the color change process, or a prolonged delay in the color change process. Non-delay to delayed timing can range from 0.01 seconds to over 36 months, such as from 0.1 second to 24 months and including from 1 minute to 12 months, e.g., between 1 hour and several months. The actual desired delay timing and mechanism for incorporating the delay will depend upon specifications for the intended product.

Timing delay mechanisms can include, but are not limited to chemical means, physical means, construct means, product design elements alone or in combination. Physical duration delay mechanisms can include condition isolation methods, laminates, overlays, blocking methods, over-print layers, additives, sealing/unsealing methods, film overlays, or a combinations there of.

By way of example, but not limitation, blocking constructs are of particular interest since they can be used in the initiation development process of a mixed co-topo-chemical duration monitoring composition. Structures intended solely to protect the duration monitoring composition and maintain its uninitiated state will find use as a duration initiation feature. Removing a barrier, blocker, inhibitor or the like that will enable the duration monitoring process is particularly relevant to a wide range of product use applications including but not limited to consumer products, industrial products, products being processed, products produced at franchises such as bakeries and dairies, products that need to be utilized in a particular time frame to ensure intended usage or timing of usage before expiration or the like.

More specifically, overlaying films or layers can be readily removed to initiate (actuate) the process of duration dependent color development in a duration monitoring composition comprising a indicator. Actuating films can include a wide range of different material types including, but not limited to plastics, synthetics, metallized plastic films, metallic films, resin films, printed films, laminated films, paper layers, coated paper layers, Mylar, wax films, fabrics, woven and nonwoven layers, rubber films, stretchable and non-stretchable films and the like.

Package components can comprise a time initiation or delay barrier. Packaging materials including thermoformed packaging constructs, injection molded packaging elements, pressure sensitive labels, film overlays, packaging sealing materials, metallized films, cellophane film wrapping materials used to seal packaged products, transparent or opaque packaging materials, paper or plastic packaging materials, printed inks on packaging components, metal compositions, or the like can be used to inhibit the time dependent color development process for a duration composition. Opening, manipulating, altering, tampering with, package placement, product utility, product removal or the like can all be used as a means of initiating color development/duration monitoring process. Conveniently the opening and closing process of unsealing and resealing a product can be used as the initiation and/or triggering mechanism for starting the duration monitoring or end-of-use indication process.

Timing/duration inhibition barriers can be in direct contact with, juxtaposed to, integrated into, distal to or separated from a duration monitoring composition. The distance between the inhibition or triggering barrier can vary, ranging from in direct contact with the composition to over 36 inches away from a duration monitoring composition, such as from direct contact to 12 inches from the composition, e.g., from direct contact and 1 inch from the composition. The determined distance between a barrier and a duration monitoring of composition will depend greatly upon the product and indicator application of interest.

Blocking film layers that, when removed, initiate the duration monitoring process include but not limited to plastics such as: polyvinyl chloride (PVC), various polyolefins such as polypropylene and polyethylene, high density polyethylene (HDPE), low density polyethylene (LDPE), cross-linked high-density polyethylene (XLPE), softened acrylic, ABS, thick Kapton™ tape materials, Teflon® (polytetrafluoroethylene (PTFE), tetrafluoroethylene TFE and fluorinated ethylene polyproplyene FEP)-based materials, brand names such as Kydex, high to low impact polystyrene, thermoplastic polyesters, nylon, styrene-butadiene, epoxy casts, polybutylene, TPX (poly(methyl pentene), terephtalate polyethylene (PET), PETE, PETF, polyethylene teraphthalate G copolymer (PETG), polysulfone (PSF), polyutethane (PUR) Thermanox™ (TMX), polymethylmethacrylate, and the like. Strong flexible plastics such as polycarbonate are often desirable. Polycarbonate can be thermoformed, pressure formed, and injection molded.

Other exemplary plastics may include, but are not limited to: ethylenechlorotrifluoreethylene (ECTFE), ethylentetrafluorethylene (ETFE), polinvinylidene fluoride (PVDF), ethylene-propylene rubber (EPR), silicone rubber (SI), Alcryn® thermoplastic rubber (TPR), HT thermoplastic rubber (HTPR), Santoprene® thermoplastic rubber (TPR), LSOH crosslinked compounds, LSOH thermoplastic compounds, methylvinyletherfluoralkoxy (MFA), perfluoroalkoxy (PFA), thermoplastic polyester elastomer (TPE), polyimide (Kapton®), polyurethane (PUR), polyvinyl chloride 105° C. (PVC), polyvinyl chloride 70° C. (PVC), low temperature polyvinyl chloride (LTPVC), oil resistant Polyvinyl chloride (OR PVC), semirigid polyvinyl (SR PVC), polyvinyl chloride polyurethane (PVC PUR), and the like. Expanded or foamed plastics can be used to increase or decrease thermal contact accordingly. Expanded plastics can be formed with a variety of different expanding agents including Expancel™ or the like.

Actuating films can be further modified for a particular performance criteria. Modifications can include active agents that endow a film with the protective qualities that provide for efficient and desired blocking. Duration time delay sensor configurations can be designed in a variety of convenient configurations including but not limited round, square, rectangular or other geometric species. Duration delay device configurations can include thick or thin substrates ranging in thickness from 0.001 millimeter to greater than 3 centimeters, such as from 0.01 millimeters thick to 1 centimeter and including from 0.1 millimeter to 0.5 millimeters thick.

Duration delay actuating films can include thick or thin dimensions ranging in thickness from 0.001 millimeters or less to greater than 3 centimeters, such as from 0.002 millimeters thick to 1 centimeter, including from 0.005 millimeter to 0.5 millimeters in thickness. The exact thickness will depend on the intended actuation characteristics, device application, cost considerations and the like

### Multi-Plexing and Multi-Parameter Monitoring Devices, Composition, and Response Indicators:

Existing temperature and environmental monitoring devices can be modified to include the disclosed duration monitoring co-topo-polymeric compositions for time duration monitoring to create products with a plurality of monitoring and environmental sensing properties. By way of example, a “Pop-Up” temperature indicator (Volk Enterprises, Inc. Turlock Calif., USA) can be modified to include the disclosed enabling composition on the external stem portion of a Pop-Up such that duration indicating composition acts as low time indicator during storage of a meat, poultry or other food product that includes the Pop-Up device. The Pop-Up device can conveniently act as an anchoring point for the duration indicator as well as a means to keep the low temperature indicator in thermal contact with the food product.

Refrigerator thermometers, analog and digital meat thermometers, disposable cooking sensors, and various other temperature indicators can be modified to contain a duration indicator comprising co-topo-polymeric compositions for time indication.

Co-topo-polymeric duration indicating compositions disclosed here within can be readily utilized with food service label materials such as dissolve-away labels utilized in food handling (DayMark Corporation, Bowling Green Station OH, USA). The duration indicating topo-polymeric indicating compositions can be conveniently imprinted on a dissolve-away label substrate and attached to a food product or carrier of chilled food products such that its activity can be monitored for temperature indication as well as be disposed of as the dissolve-away label is removed during cleaning or the like.

A range of TTI (time-temperature indicators) can be multi-plexed with or adjoined with devices and indicators utilizing duration only indicating co-topo-chemical polymerization indicators. Devices can include, but are not limited to those that are sold commercially such as and by way of example ATI, Ciba, Avery, Patel, DayMark, and the like.

A portion or region on such devices can be modified with the disclosed co-topo-polymeric duration indicating compositions such that an additional sensing element can be included in current commercial or development based products.

### Time Delays Using Recessed Configurations:

Recessed duration delay configurations can be used in combination sensors whereby the same sensor can be used for duration measurement modification. By way of example, a recessed device can have at it lowest inner indented region a printed area containing a time monitoring co-topo-chemical color change composition. The recessed device portion of the product can be made using a thermoformable polystyrene that is heat stressed during the thermoforming process.

Thermal/time delay mechanisms can include configurations that place the low sensing composition recessed within the thermal mass of a product whose duration and end-of-use is being monitored. The device configuration can be indented such that the sensing region is below the surface plain of the product. Configurations can include, but are not limited to thermoformed indents, injection molded indents, pressure formed indented devices, and other molded or manufactured parts that provide for good thermal contact with a product such that the sensing region is recessed internally to the product.

The recession depth into a product being monitored can range from 1.0 millimeters to 10 centimeters, such as from 2.0 millimeters to 5 centimeters, including from 3 millimeters to 3 centimeters, e.g., from 0.5 centimeters to 1.5 centimeters.

### Indicating Substrate Compositions:

Indicating substrate compositions include but are not limited to paper, plastic, hard surfaces, soft surfaces, stiff or rigid surfaces, compliant surfaces, printed surfaces, printable surfaces, transparent surfaces, semi-transparent surfaces, opaque surfaces, non-transparent surfaces, and the like. A substrate composition can be comprised of thick or thin materials ranging in thickness from 1 nanometer to 100 centimeters, such as from 10 nanometers to 10 centimeters, including from 1 micron to 1 centimeter, e.g., from 10 microns to 5 millimeters, including from 0.1 millimeters to 1.0 millimeters.

In some instances, it is desirable to utilize chemically active substrates for compositions such that the composition's time monitoring capabilities can be further modified by interaction with a chemically active component embedded in the substrate.

Substrates of interest include both porous and non-porous substrates.

### Combination Co-Topo-Polymeric Compositions and Other Chromogenic Compounds:

Co-topo-polymeric compositions of the invention can be used alone, in direct contact and formulation with other chromic change agents, or in adjacent combinations with other chromic change agents. Other chromic change agents include, but are not limited to other thermochromic, photochromic, mechanochromic, solvatochromic, chemochromic, biochromic, pressure chromic, piezochromic, bio-degradation product initiated indicators, optical indicators, nano sensing compositions, pH indicators, environmental sensing compositions or the like. Importantly, the combination of the indicating means of a co-topo-polymeric composition can be utilized synergistically with one or more other chromic change agent to enable a multi-parameter indicating system.

Thermochromic dyes and colorants can be added to the composition formulation to serve as an indicating means to show that a particular composition has been temperature activated for optimal use. Temperature ranges for added thermochromic transitions can be below freezing to above boiling depending on the intended use of the thermochromic composition application. Thermochromic dyes can find use in a variety of compositions and applications and formats. Thermochromic dyes can include but are not limited to compounds including: bis(2-amino-4-oxo-6-methylpyrimidinium)-tetrachlorocuprate(II); bis(2-amino-4-chloro-6-methylpyrimidinium)hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (Chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the Photoproduct 7,7adihydro-4,7,7,7a-tetramethyl-2-phenylbenzo[b]thiophene-5,6-dicarboxylic anhydride, and the like.

Other thermochromic dyes of interest include leucodyes including color to colorless and color to color formulations, vinylphenylmethane-leucocyanides and derivatives, fluoran dyes and derivatives, thermochromic pigments, micro and nano-pigments, molybdenum compounds, doped or undoped vanadium dioxide, indolinospirochromenes, melting waxes, encapsulated dyes, liquid crystalline materials, cholesteric liquid crystalline materials, spiropyrans, polybithiophenes, bipyridine materials, microencapsulated, mercury chloride dyes, tin complexes, combination thermochromic/photochromic materials, heat formable materials which change structure based on temperature, natural thermochromic materials such as pigments in beans, various thermochromic inks sold by Securink Corp. (Springfield, Va.), Matusui Corp., Liquid Crystal Research Crop., Thermographic Measurement Corporation (TMC), Segan Industries, Inc., Color Change Corporation, or any acceptable thermochromic materials with the capacity to report a temperature change or can be photo-stimulated and the like. The chromic change agent selected will depend on a number of factors including cost, material loading, color change desired, levels or color hue change, reversibility or irreversibility, stability, and the like.

### Inhibiting or Augmenting Additives:

Inhibiting and/or augmenting agents can be added or admixed to a duration monitoring composition. Additives can include, but are not limited to agents that interfere with and inhibit the color development process or augment and promote or accelerate the color development process. By way of example, BHA, BHT, salts, sugars, initiators, UV inhibitors, polymerization promoters, catalysts, inorganic or organic compounds, polymers, surfactants, diluents, stimulating compounds, environmentally sensitive compounds, ligands, nano particles, colloidal particles, and the like.

Cholesteric liquid crystals can be added as augmenting agents that affect the properties of oil based diacetylenic monomers and mixed monomer systems. The nematic and smectic phase crystallization properties of liquid crystals can be utilized to affect the topo-chemical crystallization/polymerization properties of diacetylenic compositions.

Various oils including organic, natural, inorganic, and synthetic oils can be added to diacetylenic compositions to up time shift or down time shift the original diacetylenic composition's intrinsic time threshold. Oils can include, but are not limited to corn oil, various vegetables oils, nut oils, root oils, herbal oils, paraffin oils, greases, animal fats, natural extract oils, flavor based oils, aromatic based oils, industrial oils, and the like can be added to assist in modulating the time setting of an co-topo-polymeric composition or pure diacetylenic composition.

Modulating additives or the like can be added at percentages that promote a desired duration shift response of interest. Modulating additives can be added and can be effective from 0.0001% by weight to greater than 90% by weight, such as from 0.001% up to 80% by weight, including from 0.01% up to 70% by weight, e.g., from between 0.1% to 50% by weight, such as from between 1% and 25% by weight to a soluble monomer composition.

Of interest in some instances are resettable oils, e.g., resettable oils that include a security feature of residual indicating polymer.

Alternative thermochromic materials can be utilized including, but not limited to: light-induced metastable state in a thermochromic copper (II) complex Chem. Commun., 2002, (15), 1578-1579 under goes a color change from red to purple for a thermochromic complex, [Cu(dieten)₂](BF4)2 (dieten=N,N-diethylethylenediamine); encapsulated pigmented materials from Omega Engineering Inc.; bis(2-amino-4-oxo-6-methyl-pyrimidinium) tetrachlorocuprate(II); bis(2-amino-4-chloro-6-methylpyrimidinium)hexachlorod-icuprate(II); cobalt chloride; 3,5-dinitro salicylic acid; leuco dyes; spiropyrenes, bis(2-amino-4-oxo-6-methylpyrimidinium)-tetrachlorocuprate(II); bis(2-amino-4-chloro-6-methylpyrimidinium)hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (Chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the Photoproduct 7,7adihydro-4,7,7,7a-tetramethyl-2-phenylbenzo[b]thiophene-5,6-dicarboxylic anhydride, and the like. Encapsulated leuco dyes are of interest since they can be easily processed in a variety of formats into a plastic or putty matrix. Liquid crystal materials can be conveniently applied as paints or inks to surfaces of color/shape/memory composites.

Thermochromic color to colorless options can include by way of example, but not by limitation: yellow to colorless, orange to color less, red to colorless, pink to colorless, magenta to colorless, purple to colorless, blue to colorless, turquoise to colorless, green to colorless, brown to colorless, black to colorless. Color to color options include but are not limited to: orange to yellow, orange to pink, orange to very light green, orange to peach; red to yellow, red to orange, red to pink, red to light green, red to peach; magenta to yellow, magenta to orange, magenta to pink, magenta to light green, magenta to light blue; purple to red, purple to pink, purple to blue; blue to pink; blue to light green, dark blue to light yellow, dark blue to light green, dark blue to light blue; turquoise to light green, turquoise to light blue, turquoise to light yellow, turquoise to light peach, turquoise to light pink; green to yellow, dark green to orange, dark green to light green, dark green to light pink; brown and black to a variety of assorted colors, and the like. Colors can be deeply enriched using fluorescent and glow-in-the-dark or photo-luminescent pigments as well as related color additives.

Reversible and irreversible versions of the color change agent can be employed depending on the desired embodiment of interest. Reversible agents can be employed where it is desirable to have a multi-use effect or reuse the color change effect. For example, products with continued and repeated use value will find utility of a reversible color change component comprising the final embodiment. In such instances, one may utilize a reversible thermochromic or luminescent material which can be repeated during usage. In another example, it may be desirable to record a single color change permanently. In this case, it may be desirable to utilize a thermochromically irreversible material which changes from one color to another giving rise to a permanent change and indicating that the composition should be discarded after use.

Luminescent or fluorescent pigments can be used in conjunction with co-topo-chemical polymerization compositions. Non-visible spectrum fluorescent dyes can be obscured by an one color of a diacetylenic composition or other thermochromic dye such that when a time triggering event occurs, the fluorescent signal becomes visible when utilizing the corresponding wavelength to reveal the fluorescent dye composition.

Optical Pattern and/or Message Development:

Optical patterns can be developed under triggering conditions using optical color change dye systems in combination with modeled substrate surfaces. An image can be generated by applying a pressure indicating film over a substrate layer that has been pre-surface textured or patterned. As durations are prolonged the dye layer initially comes in contact with the close proximity regions or features of the patterned substrate surface. An initial color change will occur in the dye layer that emulates the upper surfaces of the substrate. As time continues to increase, the dye layer may be forced in contact with lower regions of the substrate surface texture. Images or patterns can appear differentially as a result of the final time induced between the time indicating dye layer and the patterned or textured substrate. Partial images can be made to occur at lower times. More complete or developed images or messages can be made to appear at medium pressures. Fully developed images or completed messages can be made to appear at final desired induced duration.

### Passive and Active RFID Time Integrating Devices:

Disclosed here with-in, energies generated in RFID circuits can be utilized as a stimuli to selectively and locally induce an optical change in color change compositions. Highly sensitive and responsive duration indicating compositions can be printed selectively and adjoined with a passive or active RFID devices such that radio wave stimuli sent to the RFID device can be utilized to induce a color change dependent response. The response can be triggered in the RFID circuit for the purpose of adding a visual indication means to the RFID device which otherwise, would not only be visible during and RFID tag usage event.

### Encapsulation:

Co-topo-polymeric compositions can be co-encapsulated with existing chromic change agents, mixed with micro-encapsulated agents, separately micro-encapsulated or be utilized in combination with a second chromic change composition. How a co-topo-polymeric composition is utilized along alone or in combination with a second chromic change composition will depend on the application of interest, operating conditions, elements to be measured and the like.

A co-topo-polymeric indicating composition can be prepared using an encapsulating coating that responds to particular pressures of interest by adjusting the coating thickness and coating type. Either acid/base color change systems, donor acceptor color change composition systems, activator/dye systems, or charge transfer color change composition systems were encapsulated using standard leuco dye encapsulating processes. The hardness, strength, integrity and pressure fracture properties of the encapsulation compositions were selected to match a particular pressure level of interest. Encapsulation coatings and processes were further adjusted as to not interfere with the chemical color change properties of the color change system. Either both dye par members could be encapsulated of only a single dye pair member of a system needs to be encapsulated. In either case each dye pair member must be separated from the other member prior to utilization during time exposure.

Encapsulated dye systems can be further processed into ink or coating formulations including a liquid carrier medium that is compatible with the suspension and stabilization of an encapsulated dye, a binder for adhering the dye system to a substrate, and any necessary stabilizing components needed to mitigate any unwanted dye migration and unwanted color development. Emulsifier and surfactants can be further added to improve coating and flow characteristics during application of the dye ink to a substrate layer. Processed coating solutions can be adhered directly to a substrate, film, surface, bottom substrate layer or other convenient surface intended as part of a finished pressure indicator.

Chromic change/indicating dyes can be added in from 0.001% by weight to 80% by weight, such as form 0.01% to 50% by weight, including from 0.1% to 25%, e.g., from 0.5% to 5% by weight.

### Colorants and Pigments:

Fluorescent dyes and pigments can find use in various product applications and mediums and formats to improve the coloration of the initial product as well as acting to create a strong contrast in the composition matrix indicating that a contaminating species has been transferred into the matrix. Fluorescent dye compounds can include, but are not limited to: fluorescein, fluoresceine, resourcinolphthalein, rhodamine, imidazolium cations, pyridoimidazolium cations, dinitrophenyl, tetramethylrhodamine and the like. A wide range of fluorescent dyes that can be activated at various wavelengths and emit light at lower wavelengths can be purchased from Dayglo Inc., Swada Chemical, Sigma-Aldrich (Saint Louis, Mo.) or Molecular Probes (Eugene, Oreg.).

### Homogeneous Oil Droplet and Micro-Particulate Dispersions:

Uniform and homogeneously dispersed micro, nano, macro colloidal units can be utilized to assist in more homogeneous and tighter distribution of time transition behaviors of topochemically polymerized composition compared with compositions that have a range of distribution of micro-crystalline sizes. In general, it is anticipated that uniform crystalline micro and nano-crystalline sizes provide for a more stochastic conformational changes at specifically selected and/or desired time triggering points.

Dispersion sizes of colloidal, nano and microcrystals of co-topo-polymeric compositions can range in size from 1000 microns to 0.001 microns, such as from 500 microns to 0.01 microns, including from 250 microns to 0.5 microns, e.g., from 100 microns to 1.0 microns.

Dispersion methods can include, but are not limited to simple mixing, vortex mixing, sheer mixing, sonication, ultra-sonication, blade mixing, shaking, orbital shaking, stirring, vibratory mixing and the like. Mixing can be accomplished in durations that lead to consistent homogeneous dispersions or mixed heterogeneous and homogeneous solutions. Mixing times may range from 100 hours to 0.1 second, such as from 10 hours to 1 second, including from 10 hours to 10 seconds, e.g., from 1 hour to 1 minute. The exact energy and time delivered to the composition will depend not only on the composition, but on the final desired species size and homogeneity desired.

### Dispersion Compositions and Printing Vehicles and Additives:

Dispersive and printing vehicles and additives can comprise constituents that conveniently assist in generating highly uniform and dispersed co-topo-polymeric compositions. Dispersion compositions and printing vehicles may be formulated to have the properties of adequately dispersing the composition of interest, providing a convenient and acceptable printing and binding vehicle to a substrate of interest, and preserving the binding, crystallization, and polymerization characteristics of interest. Likewise, a dispersive/printing vehicle should provide expediency of crystallization.

Of interest is maintaining the duration monitoring co-topo-polymeric composition in a stable soluble state prior to printing and initiation of actuation. Importantly monomers will remain inactive in an amorphous non-crystalline state, in a protected un-actuated state, or in an inhibited/inactive state. In all cases, the initial state of the composition can be selected depending on the product application of interest, convenience and efficiencies during production, the product format of interest, and the desired visual and/or machine readable format to be utilized within the context of the indicator.

### Nucleators for Crystal Homogeneity and Crystallization Control:

Nano-, micro-, or macro-nucleation additives can be based on substrate-monomer interaction, monomer-nucleator interaction, a combination of interactions between both the substrate and nucleator. Nucleating compositions can include, but are not limited to: micro-particulates, silicon oxides, lake dyes, dye pigments, ground inorganic materials, salts, minerals, diatomaceous earth, oxides, gels, chromatographic resins, resins, starburst dendrimers, carbon, nano-tubes, nano particles, cellulosic particles, glass, fine metal particles, structured surfaces, structured substrates, ink pigments, vapor deposited compositions, polymeric resins, and the like.

Nucleating agents can be added at percentages that promote a desired nucleation/crystallization response of interest. Nucleating agents can be added and can be effective from 0.0001% by weight to a soluble monomer composition to greater than 90% by weight, such as from 0.001% up to 80% by weight, including from 0.01% up to 70% by weight, e.g., from 0.1% and up to 50% by weight, such as from 1% and 25% by weight.

Nucleator sizes can be of use for controlling and facilitating crystallization processes. Nucleator sizes can range from 1000 microns to 0.001 microns, such as from 500 microns to 0.01 microns, including from 250 microns to 0.5 microns, e.g., from 100 microns to 1.0 microns.

Various nucleation methods, sequences, composition formulation procedures and the like can be employed for facilitating and formulating utilizing a nucleating agent. Nucleating agents can be added or admixed during various stages of ink preparation. Nucleating agents can be added as an initial component where by co-topo compositions can and printing vehicle components can be added subsequently. Nucleating agents can be added to pre-mixed ratios of monomeric materials intended for co-topo polymeric compositions. Alternatively, nucleating agents can be added post formation of a co-topo composition that is intended as a fully functional printing vehicle whereby on the nucleating agent must be further added. Likewise, the nucleating agent can be pre-printed on an intended printing substrate such that the nucleating agent is part of a printable layer that the co-topo polymeric composition is intended to be subsequently printed on. Intimate contact between the nucleating agent and the co-topo composition should be facilitated in any of the above examples.

### Combination Homogeneous Dispersion-Nucleating Agents:

A combination of utilizing homogeneous dispersions and nucleating agents have a combined synergistic advantage of generating a narrow distribution of duration indicating transitions for a given topo-chemical polymerization composition. Likewise, one or more nucleating agents can be employed with a particular co-topo-chemical polymerization competition.

### Adherent Putty Formats

Of interest in certain embodiments are compositions that have an adherent putty format, such that the composition may be readily applied to a product of interest or substrate.

### Annealing Conditions:

Optimal crystallization conditions for monomers and mixed monomer systems can dictate and play an important role in determining the crystal quality, topo-chemical polymerization characteristics, and resultant timing duration color development transition of a monomer composition, Annealing processes can be utilized to ensure optimal control over crystal quality. Of particular interest are oil-based compositions of topo-chemically polymerizable monomer materials. Room temperature oils may undergo various phase transitions from a completely fluid-liquid oil phase to a smectic crystal to an order crystalline phase.

Oil-base diacetylenic monomers exhibit properties similar to liquid crystals used through out technology based products. Liquid crystals are partly ordered materials, somewhere between their solid and liquid phases. Their molecules are often shaped like rods or plates or some other forms that encourage them to align collectively along a certain direction. The order of liquid crystals can be manipulated with mechanical, magnetic or electric forces. Finally, liquid crystals are temperature sensitive since they turn into solid if it is too cold, and into liquid if it is too hot.

### Temperature Adjustment Formats:

Modulation, adjustment, tuning, increasing or decreasing, augmenting, phase shifting, elevating or descending, altering phase diagrams or the like of temperature of co-topo-polymeric compositions can be accomplished by mixing ratios of 2 or more analogs as disclosed herein. Various points in the process of producing a time indicating composition, marking, label, or device can be utilized for adding an analog or combining 2 or more analogs such that a final time setting is achieved. By way of example, but not limitation, forms of co-topo-polymerizable monomers can be pre-mixed prior to further processing and at the initial stage of formulation from in their neat or pure form from 0.001% of a first component to 99.999% of a second component, such as from 0.01 of a first to 99.99% of a second component, including from 0.1% of a first component to 99.9% of a second component, e.g., from 1% of a first component to 99% of a second component. The exact ratio of each component will be dictated by the desired timing setting and characteristics of the intended product application for the composition. Likewise, ratios of two or more monomers can be established using standard approaches for determining phase diagrams and time threshold curves. Complex ratios of 3 or more individual monomers can further be achieved by the same approach.

Subsequent to pre-mixing monomer ratios, the mixed compositions, providing a pre-determined timing threshold, can be added to a printing vehicle or become an initial component of a formulation that is intended to become a final printing vehicle. Likewise, in certain cases, the pre-mixed composition can be used directly on a substrate and utilized without further alteration with the exception of applying the mixed composition to the substrate.

In a second example, pre-determined ratios of selected monomers that are intended to comprise a final composition can be added at a different stages of a formulation or production process. A first component can be added in the process of ink/vehicle formulation where as a second component can be added at a later stage in the formulation, to a substrate that has been pre-printed with a first component, or at any other convenient point in the process of making a time indicating device. Importantly, final processing must provide for a means to ensure that the intended ratios of monomers admix or come in contact with one another to provide for the co-mixing effect.

Mixed co-topo-polymeric compositions provide for on-demand timing adjustments where-by an initially printed and exposed single component can be post-modified by subsequently printing a second component such that the second component comes into direct contact with part or all of the printed area of the first component. The volumes and concentrations of each component should be pre-determined such that the final ratios represent the ratios required for a pre-selected time setting based on predetermined phase diagram tests. Patterns or gradients can be generated in either the first printed component or the second printed component such that an intended duration gradient or patterned graphic results during the time triggering process. For example, a simple strip can comprise 100% of one component at one end and 100% of a second component at the other end. A gradient of composition ratios can be created from on to another either in a continuous manner or in a step function. In either case, the gradient from one end shall represent an initial low timing threshold where as the gradient at the other end will represent a final prolonged threshold. Thermometer strips can be prepared and printed such that a color change will occur geometrically along the strip as exposure timing is increased from the initial triggering initiation to the final time.

Additional details regarding fabrication protocols of interest are disclosed in U.S. application Ser. No. 12/505,405 filed on Jul. 17, 2009, the disclosure of which is herein incorporated by reference.

### UV/Liquid Crystal Temperature Modifications:

Liquid crystals and cholesteric liquid crystals, can be utilized in conjunction with co-topo-polymeric compositions and in combination with UV treatment to further adjust, modify, manipulate, up shift, down shift, or change a pre-determined time setting for a given co-topo-polymeric composition. It is known in the art that liquid crystals can be modified in their temperature settings by exposing pre-printed materials to ultra-violet light intensities. By doping liquid crystal compositions in co-topo-chemical compositions, the UV dependent temperature adjustment that affects the liquid crystal composition can be utilized to further facilitate and modulate the transition of the admixed co-topo-polymeric composition.

Such post printing modifications find use for further changing intrinsic time setting of the co-topo-polymeric composition. Likewise, the post UV processing step takes advantage of existing printing and UV exposure processes utilized by the high volume printing industry.

Practical UV intensities for modifying liquid crystals can range from 0.1 watt per square inch to over 10,000 watts per square inch, such as from 1 watt per square inch to 5,000 watts per square inch, including from 100 watts per square inch to 2,500 watts per square inch necessary to modulate the physical/chemical composition and proportionately the temperature modulating effect of a liquid crystal component in the composition that in turn effects the temperature setting of a co-topo-polymeric composition.

### Utility

Duration monitoring compositions of the invention find use in a variety of different applications. As such, applications for duration monitoring/end-of-use indication co-topo-polymeric compositions of the invention and related activity modulating agents can range in application scope from early stage production, manufacturing, or synthesis stages through to end-of-use indication of a given product, where a product or good being monitored using an indicator or composition has already expired and is no longer of any further utility or value.

Duration monitoring/end-of-use indicating co-topo-polymeric compositions can find use for a wide range of time frames, sensing, indicating, measurement, marking, inventory-management, holding composition monitoring, safety, sensitizing, industrial, food, pharmaceutical, logistics including cold chain and non cold chain uses, processing, consumer product shelf-life, fragrance release end-point, materials processing, inventory control, pharmaceutical, dental products, e.g., toothbrushes, tobacco products, hygiene products, and other market and/or product applications.

Duration monitoring compositions find direct utility with and have application to monitoring and reporting storage conditions of any product, material, or object. Of interest are products, materials and objects where maintaining storage conditions of that item dictates the viability, perishability, freshness, temperature, well being, stability, condition, viability, duration in inventory, integrity, or any other general parameter that imparts the quality and status of the product, material or object.

Duration monitoring compositions for monitoring a material's, object's or product's condition can be incorporated in a device that attaches to the product of interest or is distal to the product being monitored. The indicating composition can be printed directly on the product or object or be applied in a film or device form. The application of interest will dictate the means by which the indicating composition will be utilized. For example, perishable dairy product will find use with an indicator attached to the product so that the viability and age integrity of the product can be visualized form the point of manufacturing, throughout production and further processing and packaging, through storage, through inventory, through transport and logistics, through docking and inspection, through delivery, through storage and display, through final pickup and handling by a consumer or intended user of the product or object, duration of use of a product, and an end-of-life indication for the product or object being monitored.

Duration indicating co-topo-polymeric compositions can be readily printed using a wide range of conventional and innovative printing mechanisms. Printing can be accomplished using high-speed methods such as flexographic printing, rotogravier, off-set printing and the like. Printing can also be accomplished using medium speed processes such as screen printing, rotary screen printing, and fluid application. Printing can be accomplished using ink jet printing, drop on demand printing, continuous ink jet printing, spray coating, droping methods, flood coating methods, dip coating methods, metering methods, film transfer methods, dye sublimation processes, and the like. Of interest in certain embodiments are markers or pens that contain an mount of time duration composition and may be employed to manually apply the composition to an item of interest, e.g., a produce or substrate. The exact printing method utilized will depend on the type of printing required, formulations utilized, device configurations, volume requirements and the like. Any practical, visually interpretive, understandable, alerting, and/or communicative graphic can be printed to facilitate and communicate the duration monitoring event.

Single or pure compositions disclosed herein can also be utilized alone or in conjunction with mixed systems. The exact composition and concentration utilized will depend on the time setting of interest, material attributes such as stability and color development, processing utilized, processing requirements such as solubility and printing vehicle type, augmenting agents required along with a finished ink composition and the like.

Compositions disclosed here within have been tested and analyzed for activities and function in a wide range of printing vehicles including water-based, solvent based, ultra-violet light cured, single component curable resins, dual component epoxy printing resins, sublimation resins, oil base vehicles, fast drying and slow drying vehicles, pigmented and non pigmented resins, clear and opaque vehicles, and a wide range of conventional and non conventional printing resins and the like. The exact printing vehicle utilized will depend on the type of printing required, formulations utilized, device configurations, volume requirements and the like.

Duration monitoring compositions enable the protection of products, materials, personnel, goods and the like an on-going, convenient, and direct means for determining a history or timing. Additional product, market, production, and company opportunities that find need for rapid timing monitoring include but are not limited to military goods that need to be stored at ambient temperatures, vegetables that may harbor pathogens, case ready foods that are either pre-cooked or not, disposable and reusable packaging including pre-molded or thermo formed packaging used for foods or other perishables, the packaging industry, blood bags and the medical industry for safety and storage, donated body parts for the medical industry, flowers and the flower industry for transport and storage, seeds and the seed industry, plastic shrink wrap films that need time recording during shipping and storage, beer and wine among other alcoholic beverages that require defined storage and shipping timing and/or temperature, electronic components and technology industries that require time control on specialized parts, rare chemicals and the chemical industry, biochemicals and the biotechnology industry, medications both over the counter and prescribed, dairy products including milk, cheese, and ice cream, hummus and other perishable foods, film and papers for the photographic industry, shellfish and other fish for the fish industry, expedient delivery, and the like.

Duration monitoring compositions satisfy an immediate and growing need to rapidly and accurately develop, produce and supply specific visual or machine readable timing indicators that cover a very broad duration range. The invention herein provides for the rapid ability for formulate timing indicating products that undergo a time-dependent color change at a predetermined time that may range from below 10 seconds to above 36 months, such as from 1 minute to 12 months, including from 1 hour to 6 months, e.g., from several hours to several days.

As described herein, a major enabling advantage of co-topo-polymeric compositions is that a small number of monomer structural analogs when mixed and combined together utilizing measured phase diagrams, phase curves, and/or empirical testing can be admixed and formulated to cover a wide range of product applications. Importantly and by way of example, careful adjustment of molecular, molar or weight ratios of only a selected number like-kind or related molecular analogs can be utilized to cover a time range of over several years. Either by experiment or by phase diagram prediction, co-topo-polymeric compositions can be formulated and adjusted in ratios using only 2-3 analogs to cover create indicators that cover a prolonged time range.

Device and indicators for various product applications can include single time settings and/or a plurality of individual time settings. By way of example, but not limitation, indicators can include 1 to over 100 timing zones comprised with specified co-topo-polymeric compositions with pre-determined timing settings, such as from 1 to over 50 independent or plural timing zones, including from 1 to 20 timing zones, e.g., 1 to 10 timing event settings. The number of timing settings selected to utilize ratios of co-topo-polymeric materials will depend on the product application of interest.

Formulations of duration monitoring compositions for selected indicator and product types can be as determined both experimentally and by physical chemical diagrams, can be achieved by choosing molecular features and attributes including but not limited to: hydrocarbon chain length, strait or branched chains, pure hydrocarbon chains or substituted chains, position of the diacetylenic moiety along the length of a hydrocarbon chain, number of diacetylenic moieties in a given molecular analog, number or hydrocarbon chains containing diacetylenic moieties comprising the molecular analog, the presence or absence of hydrophilic substituents, the presence of hydrogen bonding groups including, but not limited to esters, amide groups, sulfhydral groups, quaternary charged groups, other charged species, chiral and achiral groups, appended groups, multiple or single hydrogen bonding groups, non-hydrogen bonding analogs, polar and non-polar head groups, head groups with substituents or are unsubstituted, head group side chains, bis-coupled analogs where coupling is direct or through a linkers, bis molecules with different molecular compositions, and the like.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A duration monitoring composition comprising at least a first polymerizable monomer, wherein the duration monitoring composition is configured to change color at a predetermined time after polymerization initiation of the composition.

2. The duration monitoring composition according to claim 1, wherein the composition comprises at least one of:
(i) a second monomer that is distinct from the first monomer; and
(ii) an effector compound.

3. The duration monitoring composition according to claim 2, wherein the composition comprises both of:
(i) a second monomer that is distinct from the first monomer; and
(ii) an effector compound.

4. The duration monitoring composition according to claim 2, wherein the first and second monomers are diacetylenic monomers.

5. The duration monitoring composition according to claim 4, wherein first and second diacetylenic monomers differ from each other in one or more of: monomer chain length, head-group structure, bond positioning, appendages, chirality, related features, and/or combinations thereof.

6. The duration monitoring composition according to claim 5, wherein each of the first and second diacetylenic monomers is independently used to develop a color.

7. The duration monitoring composition according to claim 1, wherein the composition is configured to initiate polymerization upon exposure to ambient conditions.

8. The duration monitoring composition according to claim 1, wherein the composition is configured to initiate polymerization upon application of a stimulus to the composition.

9. The duration monitoring composition according to claim 8, wherein the stimulus comprises removing a barrier.

10. The duration monitoring composition according to claim 8, wherein the stimulus comprises exposing the composition to UV radiation.

11. The duration monitoring composition according to claim 1, wherein the duration monitoring composition is applied directly to an object to be monitored.

12. The duration monitoring composition according to claim 1, wherein the duration monitoring composition is present on a substrate.

13. The duration monitoring composition according to claim 12, wherein the substrate is a label.

14. The duration monitoring composition according to claim 13, wherein the label is applied to an object to be monitored.

15. The duration monitoring composition according to claim 12, wherein the substrate is a sensor.

16. The duration monitoring composition according to claim 1, wherein the composition further comprises a thermochromic dye.

17. The duration monitoring composition according to claim 1, wherein the composition further comprises a cholesteric liquid crystal.

18. The duration monitoring composition according to claim 1, wherein the composition further comprises an oil.

19. The duration monitoring composition according to claim 1, wherein the composition further comprises a luminescent or fluorescent pigment.

20. The duration monitoring composition according to claim 1, wherein the one or more components of the composition are encapsulated.

21. The duration monitoring composition according to claim 1, wherein the composition further comprises a nucleator.

22. A duration indicator device comprising a duration monitoring composition comprising at least a first polymerizable monomer, wherein the duration monitoring composition is configured to change color at a predetermined time after polymerization initiation of the composition.

23. The duration indicator device according to claim 22, wherein the duration monitoring composition is present on a surface of a solid support.

24. The duration indicator device according to claim 23, wherein the device comprises two or more distinct duration monitoring compositions on the surface of the solid support.

25. The duration indicator device according to claim 24, wherein the two or more distinct duration monitoring compositions change color at a different time following initiation of polymerization.

26. The duration indicator device according to claim 22, wherein the device further comprises a sensor that is not a duration monitoring composition.

27. The duration indicator device according to claim 22, wherein the device comprises the duration monitoring composition in a machine readable format.

28. The duration indicator device according to claim 27, wherein the format is a barcode.

29-31. (canceled)

32. A method of determining a time parameter of a product, the method comprising:
identifying a color change in a duration monitoring composition associated with the product, where the duration monitoring composition comprises at least a first polymerizable monomer and is configured to change color at a predetermined time after polymerization initiation of the composition; and
determining the time parameter from the identified color change.

33-61. (canceled)
